(19) United States
(12) Patent Application Publication
(10) Pub. No.: US 2008/0009470 A1
(43) Pub. Date: Jan. 10, 2008

(54) Acremonic Acid Derivatives

(76) Inventors: Gerd Ascher, Kundl (AT); Mathias Ferencic, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/578,034

(22) PCT Filed: Nov. 4, 2004
(86) PCT No.: PCT/EP04/12496

§ 371 Date: May 10, 2006

(30) Foreign Application Priority Data
Nov. 5, 2003 (GB) 0325828.2

(51) Int. Cl. A61K 31/56; A61P 43/00; C07D 215/04

(52) U.S. Cl. 514/171; 514/169; 540/2

(57) ABSTRACT

A compound of formula

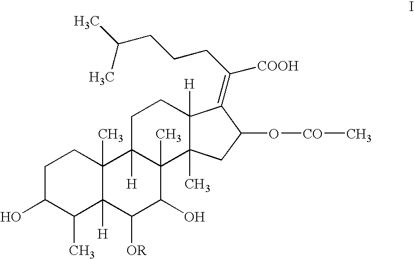

wherein
R has various meanings and its use as a pharmaceutical.

The present invention relates to acremonic acid derivatives.

In one aspect the present invention provides 2-(16-Acetoxy-3,7-dihydroxy-4,8,10,14-tetramethyl-6-[hydroxy, (C₁₋₂₂)alkoxy or carbonyloxy]-hexadecahydro-cyclopenta[a]phenanthren-17-ylidene)-6-methyl-heptanoic acids, e.g. a compound of formula

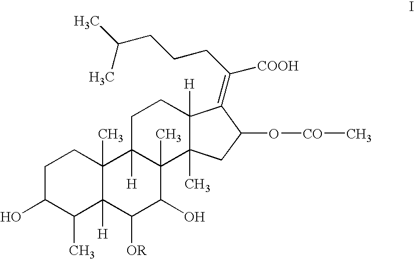

e.g. including a compound of formula

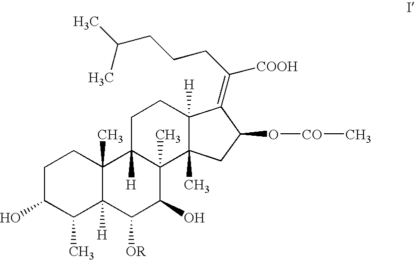

wherein
R is hydrogen, CO—R₁ or (C₁₋₂₂)alkyl, such as methyl, ethyl, n-propyl or n-hexyl, and
R₁ is hydrogen, (C₁₋₂₂)alkyl, such as ethyl, n-propyl, isopropyl, 2-ethylpropyl, 1,1-dimethylpropyl, n-butyl, isobutyl, t.butyl, n-pentyl, t.butylmethyl, n-hexyl; (C₃₋₈)cycloalkyl, (C₁₋₆)alkoxy-(C₁₋₆)alkyl, (C₁₋₄)alkoxy-(C₁₋₄)alkoxy-(C₁₋₄)alkyl, amino(C₁₋₄)alkyl, halo(C₁₋₆)alkyl, hydroxy(C₁₋₄)alkyl, hydrogencarbonyl, hydroxycarbonyl-(C₁₋₄)alkyl, (C₁₋₄)alkoxycarbonyl-(C₁₋₄)alkyl, (C₆₋₁₈)aryl, heterocyclyl having 5 or 6 ring members and 1 to 4 heteroatoms selected from S, O or N, or brigded (C₇₋₁₂)cycloalkyl;
e.g. wherein
   cycloalkyl is unsubstituted or substituted, such as unsubstituted cycloalkyl or cycloalkyl one or morefold substituted by (C₁₋₄)alkyl or (C₁₋₄)alkoxy, such as 1-methyl-cycloprop-1-yl, 2-methyl-cyclopropyl, 2,2,3,3-tetramethyl-cyclopropyl, 3-methoxy-cyclohexyl, 4-methoxy-cyclohexyl;
   amino is unsubstituted or substituted, e.g. unsubstituted or substituted by (C₁₋₄)alkyl, di(C₁₋₄)alkyl, or (C₁₋₄)alkoxycarbonyl; e.g. (C₁₋₄)alkoxycarbonyl, such as methoxycarbonyl,
   aryl is unsubstituted or substituted by amino.

Preferably in a compound of formula I
R is hydrogen, (C₁₋₆)alkyl, or CO—R₁,
R₁ is hydrogen, (C₁₋₆)alkyl, (C₃₋₆)cycloalkyl, e.g. unsubstituted (C₃₋₆)cycloalkyl or (C₃₋₆)cycloalkyl substituted by one or more halogen, methyl or methoxy; (C₁₋₃)alkoxy-(C₁₋₃)alkyl, methoxy-(C₁₋₂)alkoxy-(C₁₋₂)alkyl, aminomethyl, e.g. including methoxycarbonylamino; halo(C₁₋₄)alkyl comprising one or two halogen atoms; e.g. fluoro(C₁₋₄)alkyl, such as e.g. fluoropropyl, e.g. including fluoroisopropyl; hydroxymethyl, hydroxycarbonylmethyl, methoxycarbonyl-(C₁₋₂)alkyl, phenyl, e.g. phenyl substituted by amino, such as dimethylamino; tetrahydrofuranyl or adamantanyl.

In a compound of formula I or I_{P}, respectively, each single defined substituent may be a preferred substituent, e.g. independently of each other substituent defined.

In another aspect the present invention provides a compound of formula I, wherein R is a group of formula

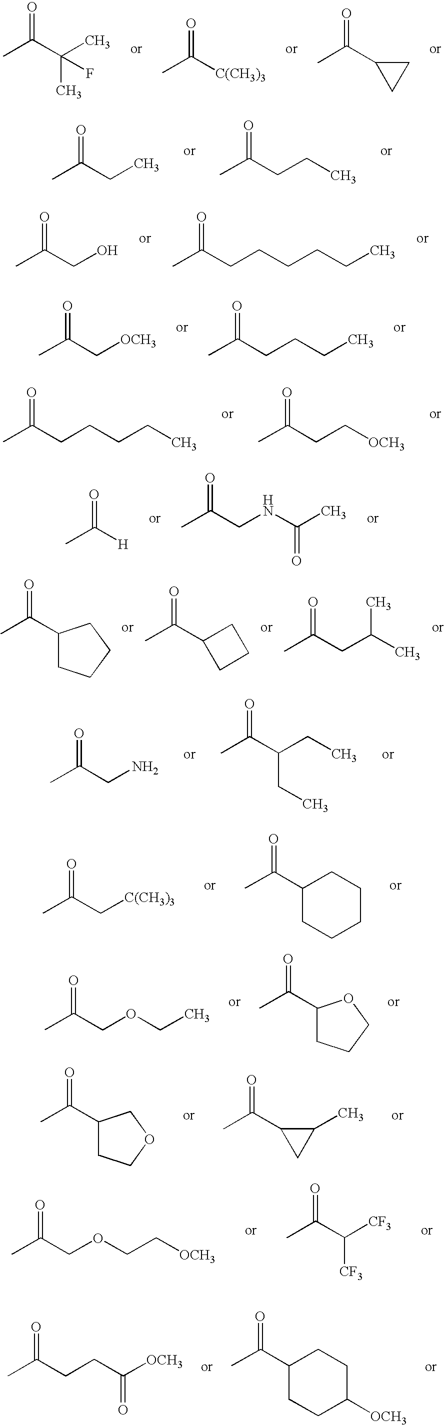

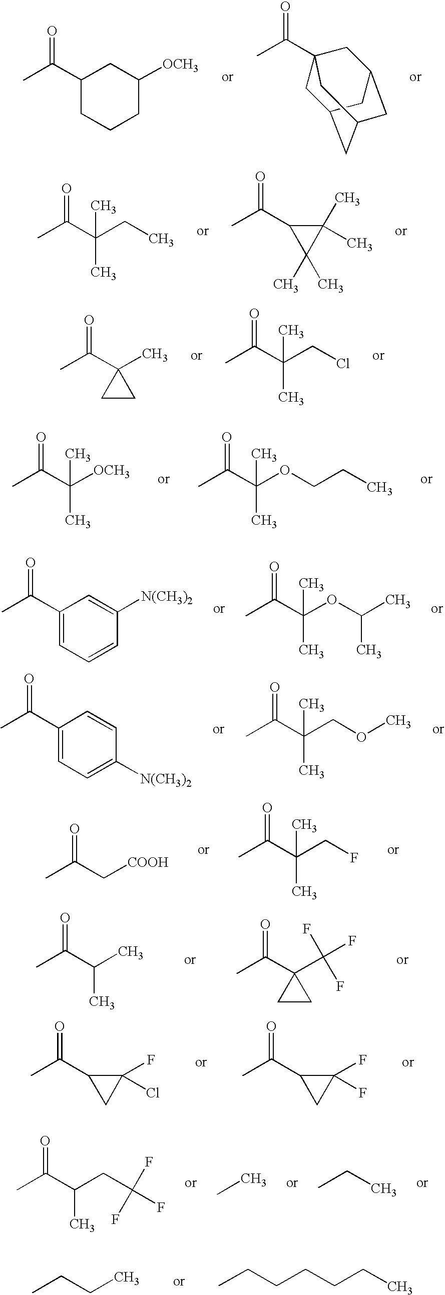

or hydrogen

In another aspect the present invention provides a compound of formula

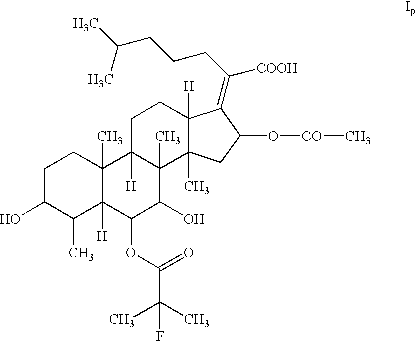

including a compound of formula

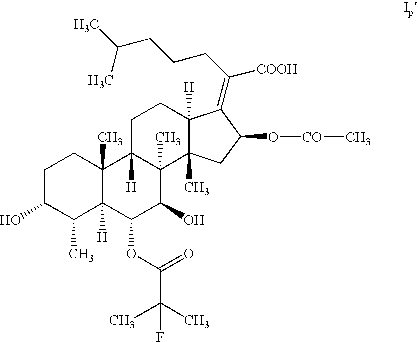

If not otherwise defined herein
alkyl includes (C₁₋₂₂)alkyl, such as (C₁₋₈)alkyl, e.g. (C₁₋₆)alkyl, e.g. including (C₁₋₄)alkyl;
cycloalkyl includes (C₃₋₈)cycloalkyl, e.g. (C₃₋₆)cycloalkyl, such as cyclopropyl, cyclobutyl, cyclopentyl, cyclohexyl:
alkoxyalkyl includes (C₁₋₆)alkoxy-(C₁₋₆)alkyl, such as (C₁₋₄)alkoxy-(C₁₋₄)alkyl, e.g. methoxymethyl, ethoxymethyl, 1,1-dimethyl-1-n-propoxymethyl, 1,1-dimethyl-1-isopropoxymethyl, methoxyethyl, 1,1-dimethyl-1-methoxy-methyl;
alkoxy includes (C₁₋₆)alkoxy, such as (C₁₋₃)alkoxy; e.g. methoxy, ethoxy, propoxy;
haloalkyl includes halo(C₁₋₆)alkyl, e.g. halo(C₁₋₄)alkyl, comprising one or more halogen atoms, e.g. including (C₁₋₄)alkyl substituted by one or more CF₃, such as —CH(CF₃)₂, 1,1-dimethyl-2-fluoroethyl, 1,1-dimethyl-2-chloroethyl or fluoro-isopropyl;
hydroxyalkyl includes hydroxy(C₁₋₄)alkyl, such as hydroxymethyl;
alkoxycarbonylalkyl includes (C₁₋₄)alkoxycarbonyl-(C₁₋₄)alkyl, such as methoxycarbonyl-(C₁₋₄)alkyl, e.g. methoxycarbonylethyl;
alkoxy-alkoxy-alkyl includes (C₁₋₄)alkoxy-(C₁₋₄)alkoxy-(C₁₋₄)alkyl, e.g. methoxy-ethoxy-ethyl;
aminoalkyl includes amino(C₁₋₄)alkyl, such as aminomethyl;
amino includes unsubstituted amino and amino substituted by (C₁₋₄)alkyl, di(C₁₋₄)alkyl, or (C₁₋₄)alkoxycarbonyl; such as dimethylamino, methoxycarbonylamino;
heterocyclyl includes heterocyclyl having 5 or 6 ring members and 1 to 4 heteroatoms selected from S, O and N, e.g. heterocyclyl having 5 ring members, e.g. the heteroatom is selected from O, such as tetrahydrofuranyl;
aryl includes (C₆₋₁₈)aryl, such as phenyl;
brigded cycloalkyl includes cycloalkyl bridged by alkyl, e.g. bridged (C₇₋₁₂)cycloalkyl, such as bridged (C₁₀)cycloalkyl, e.g. adamantanyl;
halogen includes fluoro, chloro, bromo, iodo, e.g. fluoro, chloro, e.g. fluoro.

Compounds provided by the present invention are hereinafter designated as “compound(s) of (according to) the present invention”. A compound of formula I includes a compound of formula I_{P}. A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes. A salt of a compound of the present invention includes a metal salt or an acid addition salt. Metal salts include for example alkali or earth alkali salts, e.g. a sodium salt. Acid addition salts include salts of a compound of formula I with an acid, e.g. hydrogen fumaric acid, fumaric acid, naphthalin-1,5-sulphonic acid, hydrochloric acid, deuterochloric acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of isomers and mixtures thereof; e.g. optical isomers, diastereoisomers, cis/trans conformers. A compound of the present invention may e.g. contain asymmetric carbon atoms and may thus exist in the form of enatiomers or diastereoisomers and mixtures thereof, e.g. racemates. Substituents at any asymmetric carbon atom may be present in the (R)-, (S)- or (R,S)-configuration, preferably in the (R)- or (S)-configuration. E.g., a compound of formula I has several asymmetric C-atoms and substituents bound to such asymmetric C-atoms may be in the (R)- and in the (S)-configuration, e.g. including mixtures thereof, e.g. as set out in a compound of formula I_{P}. Preferably a compound of formula I is a compound of formula I_{P}. Also a compound of formula I has a double bond and substituents bound to that double bond may be in the form of cis- or trans conformers, or mixtures thereof.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

In the following it is referred to the numbering system of the ring structure and substituents as set out in a compound of formula I below:

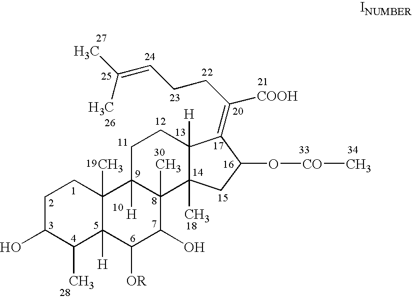

In another aspect the present invention provides a process for the production of a compound of formula I comprising the steps
a. protecting the carboxy group of position 21 and optionally the hydroxy group attached to the ring structure in position 3 of the ring structure in a compound of formula

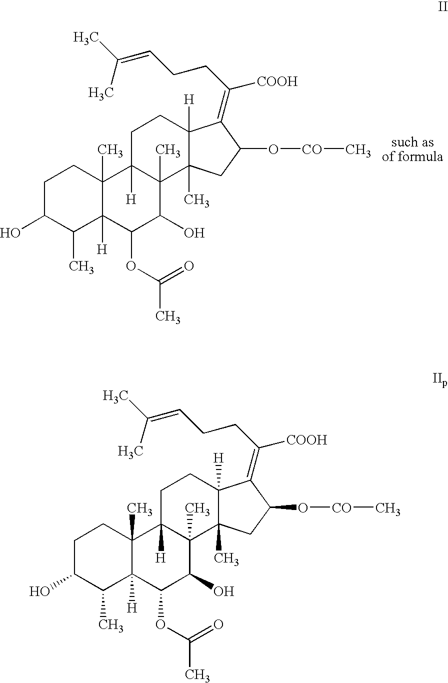

to obtain a compound of formula II, or II_{P}, respectively, wherein the carboxy group of position 21 is protected and the hydroxy group attached to the ring structure in position 3 is optionally protected,
b. splitting off the acetyl group from the acetoxy group in position 6 of the ring structure from a compound as obtained in step a., to obtain a compound as obtained in step a, wherein the group attached to position 6 of the ring structure is hydroxy,
c1. either hydrogenating the double bond in positions 24 and 25 and, e.g. in the course of double bond hydrogenation, splitting off the protecting group(s) from a compound as obtained in step b., to obtain a compound of formula I, wherein R is H, or
c2. reacting a compound as obtained in step b. with a (C₁₋₈)alkylhalogenide, hydrogenating the double bond in positions 24 and 25, and, e.g. in the course of double bond hydrogenation, splitting off the protecting group(s), to obtain a compound of formula I, wherein R is (C₁₋₈)alkyl, or
c3. reacting a compound as obtained in step a. with a compound of formula R′₁—COOH, wherein R′₁ has the meaning of R₁ as defined above, and additionally includes residues as defined in R₁, wherein functional groups, such as amino, hydroxy, carboxyl, are protected, either in the presence of a condensation agent, or with a compound of formula R′₁—COOH, wherein R′₁ is as defined above, in a reactive form, e.g. in the form of a carboxylic acid halogonide, to obtain a compound as obtained in step b., wherein the group attached to the ring structure in position 6 is a group of formula CO—R′₁, wherein R′₁ is as defined above, hydrogenating the double bond in positions 24 and 25, and, e.g. in the course of double bond hydrogenation, splitting off the protecting group(s), to obtain a compound of formula I, wherein R is a group of formula CO—R₁, wherein R₁ is as defined above, or wherein R is a group of formula CO—R′₁, wherein R′₁ is as defined above, and optionally splitting of protecting groups in R′₁, e.g. if (still) present, and
d. isolating a compound of formula I as obtained in step c. from the reaction mixture.

The protecting group attached to the carboxy group of position 21 is present, and the protecting group attached at oxygen atom attached to the ring structure in position 3, is optionally present. The reaction works in both cases, but, e.g. to obtain higher purity of the reaction products, both protecting groups are preferably present. Protecting groups include groups as appropriate, e.g. such as conventional, preferably protection groups which may be split off by hydrogenation under conditions, under which the double bond in positions 24 and 25 is converted into a single bond. Such groups e.g. include benzyloxymethyl and diphenylmethyl groups, e.g. and benzyl groups. E.g. the protection group attached to the group of position 21 is benzyloxymethyl or diphenylmethyl, and the protecting group attached to the oxygen atom which oxygen atom is attached to the ring structure in position 3, is either other than a protecting group, e.g. hydrogen or benzyloxymethyl.

R′₁ has the meaning of R₁ as defined above and additionally includes residues as defined in R₁, wherein functional groups, such as hydroxy, carboxyl and amino, are protected, e.g. hydroxy or carboxyl are protected by a benzyl group; amino is protected by a benzyloxycarbony group I; e.g. residues of R₁ having functional groups such as amino, carboxy or hydroxy, are in a protected form, e.g. in the form of benzyloxycarbonylamino, benzyloxy or benzyloxycarbonyl. Such protecting groups may be split off in the course of double bond hydrogenation in position 24 and 25, or at an appropriate stage.

In another aspect the present invention provides a process for the production of a compound of formula I, wherein R is as defined above, comprising hydrogenating the double bond in positions 24 and 25 and splitting off the protecting group(s), e.g. in the course of double bond hydrogenation, in a compound of formula

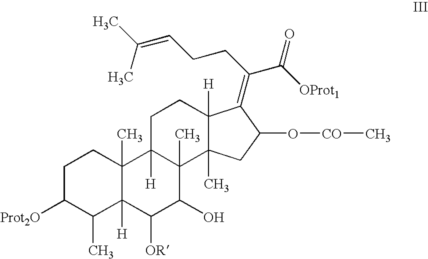

such as of formula

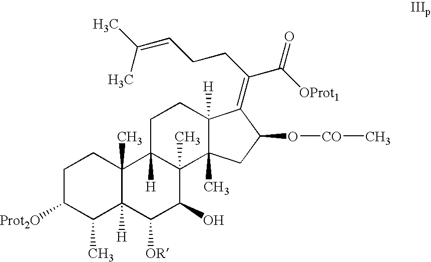

wherein
Prot₁ is a protecting group, such as benzyloxymethyl or diphenylmethyl, e.g. benzyloxymethyl,
Prot₂ is either other than a protecting group, or is a protecting group, e.g. Prot₂ is H or benzyloxymethyl, and R′ has the meaning of R as defined above and additionally includes residues as defined in R, wherein functional groups, such as amino, hydroxy, carboxyl groups, are protected.

In a preferred embodiment, a compound of formula I may be produced by a process comprising the steps
a. reacting a compound of formula II, or II_{P}, respectively, with benzyloxymethylchloride in the presence of a base, e.g. Hünig's base, in organic solvent, e.g. an halogenated hydrocarbon, such as CH₂Cl₂, to obtain a compound of formula

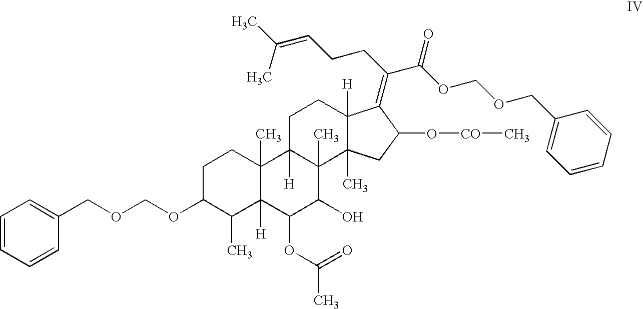

such as of formula

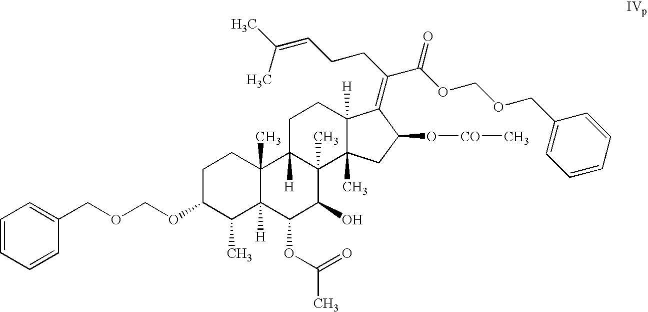

b. reacting a compound of formula IV, or IV_{P}, respectively, with a base, e.g. an alkali or earth alkali hydroxide, such as NaOH, in organic solvent, e.g. aqueous organic solvent, e.g. in a solvent mixture, such as terahydrofuran/MeOH/H₂O, to obtain a compound of formula

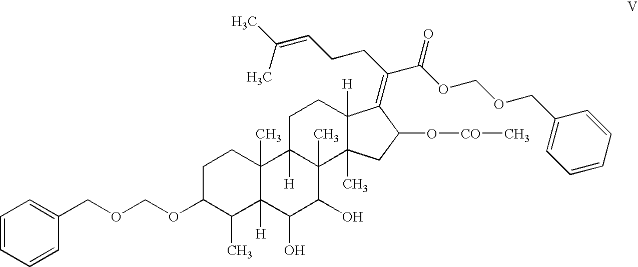

such as of formula

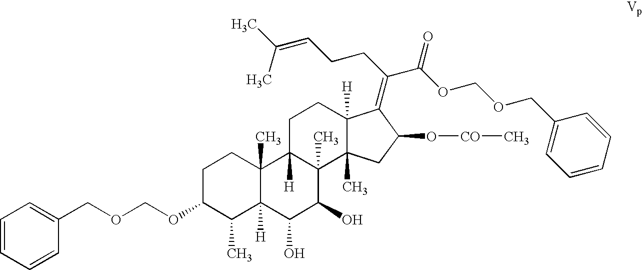

c1. reacting a compound of formula V, or V_{P}, respectively, with a compound of formula R′₁—COOH, wherein R′₁ has the meaning of R₁ as defined above and additionally includes residues as defined in R₁, wherein functional groups, such as amino, hydroxy, carboxyl, groups are protected, in the presence of a condensation agent, such as N′-(3-dimethylamino-propyl)-N-ethylcarbodiimide hydrochloride, and in the presence of a base, e.g. 4-dimethylaminopyridine, in organic solvent, e.g. halogenated hydrocarbon, such as CH₂Cl₂, or
c2. reacting a compound of V, or V_{P}, respectively, with a compound of formula R′₁—COCl, wherein R′₁ has the meaning of R₁ as defined above, and additionally includes residues as defined in R₁, wherein functional groups, such as amino, hydroxy, carboxyl groups, are protected, in the presence of a base, such as pyridine and 4-dimethylaminopyridine, to obtain a compound of formula

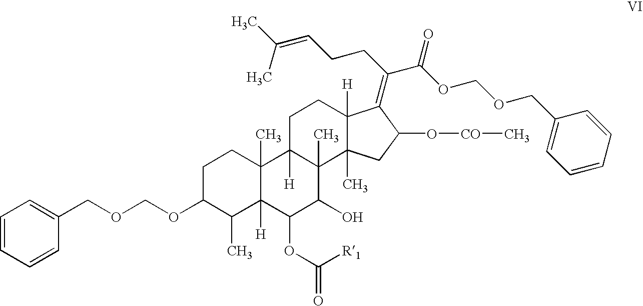

such as of formula

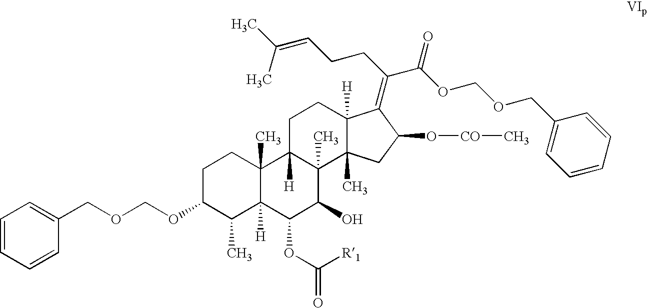

wherein R′₁ is as defined above,
d. hydrogenating the double bond in positions 24 and 25 in a compound of formula VI, or VI_{P}, respectively, e.g. by reaction with H₂, in the presence of a catalyst, such as palladium, e.g. Pd(OH)₂/C, and, e.g. in the course of double bond hydrogenation splitting off protecting group(s), and optionally splitting off protection groups in R′₁, and
e. isolating a compound of formula I, or I_{P}, respectively, wherein R is —COR₁, and R₁ is as defined above as obtained in step d. from the reaction mixture.

In another aspect the present invention provides a compound of formula

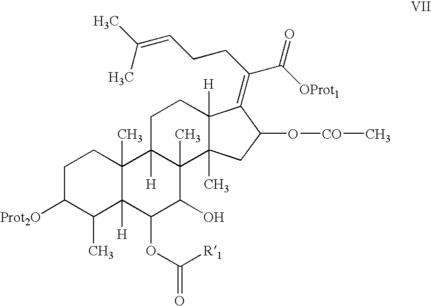

such as of formula

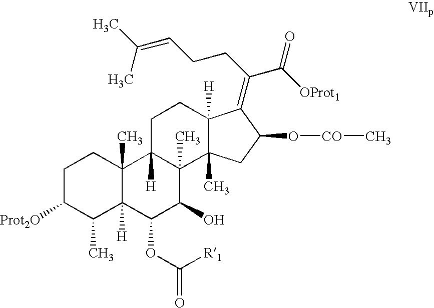

wherein
Prot₁ is a protecting group, such as benzyloxymethyl or diphenylmethyl, e.g. benzyloxymethyl, and
Prot₂ is either other than a protecting group, or is a protecting group, e.g. Prot₂ is H, benzyloxymethyl or diphenylmethyl, and R′₁ is as defined above, e.g. which compounds of formula VII, or VII_{P}, respectively, are useful as intermediates in the production of a compound of formula I, or I_{P}, respectively.

A compound of formula VII, or VII_{P}, respectively, includes compounds of formulae VI, or VI_{P}, respectively.

In another aspect the present invention provides a compound of formula IV, or IV_{P}, respectively, and of formula V, or V_{P}, respectively and of formula VI, or VI_{P}, respectively, wherein R′₁ is as defined above, which compounds are useful as intermediates in the production of a compound of formula I, or I_{P}, respectively, wherein R is a group —CO—R₁.

In another aspect the present invention provides a compound of formula

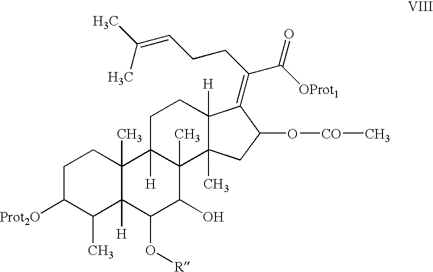

such as of formula

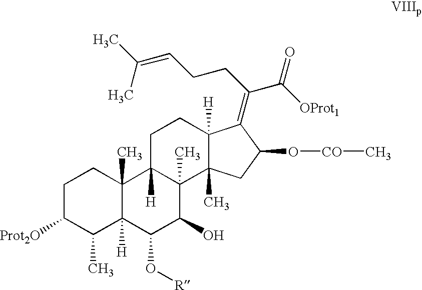

wherein Prot₁ and Prot₂ are as defined above, and R″ is (C₁₋₈)alkyl; e.g., which compounds are useful as intermediates in the production of a compound of formula I, or I_{P}, respectively, wherein R is (C₁₋₈)alkyl.

In a compound of formula VIII, or VIII_{P}, respectively, preferably
Prot₁ is diphenylmethyl,
Prot₂ is benzyloxymethyl
R″ is (C₁₋₆)alkyl, e.g. methyl, ethyl, n-propyl or hexyl.

A compound of the present invention of formulae II, II_{P}, III, III_{P}, IV, IV_{P}, V, V_{P}, VI, VI_{P}, VII, VII_{P}, VIII and VIII_{P} is herein also designated as “an intermediate of (according to) the present invention”. An intermediate of the present invention includes an intermediate in any form, e.g. in free form, in the form of a salt, in the form of a solvate and in the form of a salt and a solvate.

In another aspect the present invention provides an intermediate of the present invention in the form of a salt.

Such salts include pharmaceutically acceptable salts and pharmaceutically unacceptable salts, e.g. for preparation/isolation/purification purposes. A salt of an intermediate of the present invention includes a metal salt or an acid addition salt. Metal salts include for example alkali or earth alkali salts, e.g. a sodium salt. Acid addition salts include salts of a compound of formula I with an acid, e.g. hydrogen fumaric acid, fumaric acid, naphthalin-1,5-sulphonic acid, hydrochloric acid, deuterochloric acid.

An intermediate of the present invention may exist in the form of isomers and mixtures thereof; e.g. optical isomers, diastereoisomers, cis/trans conformers, similarly as described above for a compound of the present invention. Isomeric mixtures may be separated as appropriate, e.g. according, e.g. analogously, to a method as conventional, to obtain pure isomers. The present invention includes an intermediate of the present invention in any isomeric form and in any isomeric mixture.

The present invention also includes tautomers of an intermediate of the present invention, where tautomers can exist.

In an intermediate of the present invention beside the (Prot-) protected groups, further functional groups, where present, optionally may be in protected form, e.g. amino, hydroxy or carboxyl groups, as indicated above; or may be in the form of a salt, where a salt-forming group is present. Protecting groups, optionally present beside Prot₁ and Prot₂, may be removed at an appropriate stage, e.g. according, e.g. analogously, to a method as conventional.

A compound of formula I, or I_{P}, respectively, obtained by a process provided by the present invention may be converted into another compound of formula I, or I_{P}, respectively, e.g. or a compound of formula I, or I_{P}, respectively, obtained in free form may be converted into a salt of a compound of formula I, or I_{P}, respectively, and vice versa.

Any compound described herein, e.g. a compound of the present invention and intermediates of formula II, II_{P}, III, III_{P}, IV, IV_{P}, V, V_{P}, VI, VI_{P}, VII, VII_{P}, VIII and VIII_{P} may be prepared as appropriate, e.g. according, e.g. analogously, to a method as conventional, e.g. or as specified herein.

The compounds of the present invention, e.g. including a compound of formula I and of formula I_{P}, exhibit pharmacological activity and are therefore useful as pharmaceuticals. E.g., the compounds of the present invention show antimicrobial, e.g. antibacterial activity against gram positive bacteria and gram negative, such as Staphylococcus, e.g. S. aureus, MRSA (Methicillin Resistant S. aureus), MSSA (Methicillin Sensitive S. aureus), Enterococcus, e.g. E. faecalis, E. faecium, Moraxella, e.g. M. catarrhalis, in vitro in the Agar Dilution Test and/or Micro Dilution Test for bacteria according to National Committee for Clinical Laboratory Standards (NCCLS) 1993,
Document M7-A4, Vol. 20, No. 2, 2000: “Methods for dilution Antimicrobial Susceptibility Tests for Bacteria that Grow Aerobically”—Third Edition, Approved Standard”; and
Document M26-A, Vo. 19, No. 18, 1999: Methods for determining antibactericidal activity of antimicrobial agents,
Document M11-A3 for anaerobic bacteria,
in a concentration from about 0.1 to ca. 25.6 μg/ml, e.g. using strains including Staphylococcus aureus (ATCC 29213 and ATCC 29506); Enterococcus faecalis ATCC 29212;
and in vivo in the septicaemia mouse model, in accordance to the method description Nr. 159 A-5, approved by Austrian Health Authorities (MA 58, no. 2968/95 of Oct. 12, 1995), e.g. when administered at dosages from 0.05 to 50 mg/kg body weight.

E.g., mice infected with Staphylococcus aureus (ATCC 49951, MSSA), and treated orally 1 and 4 hours after infection with a compound of example 1, e.g. in the form of its sodium salt, show an ED50 value of ca. 8.55 mg/kg body weight (ranging from 5.54 to 13.34). Mice infected with S. aureus B29 (clinical isolate, MRSA) and treated orally 1 and 4 hours after infection with a compound of example 1, e.g. in the form of its sodium salt, show an ED₅₀ value of ca. 6.65 mg/kg body weight, (ranging from 4.25 to 11.98). Mice infected with S. aureus B29 (clinical isolate, MRSA) and treated subcutaneously 1 and 4 hours after infection with a compound of example 1, e.g. in the form of its sodium salt, show an ED₅₀ value of ca. 3.20 mg/kg body weight, (ranging from 1.93 to 5.85). The ED₅₀ values are calculated by Probit analysis of the administered dosages of compounds. Activity is determined by numbers of surviving animals per group of 8 or 6 mice, respectively, per dosage unit on day 5 after infection.

The compounds of the invention show a surprising overall activity spectrum.

It has, for example, been determined that the MIC₉₀ (μg/ml) of the compound of example 1, e.g. in the form of its sodium salt, against methicillin-sensitive Staphylococcus aureus (MSSA) strains is 0.25 μg/ml (n=26) and against methicillin-resistant S. aureus (MRSA) strains MIC₅₀ is 0.2 μg/ml (n=26). Furthermore, the compound of example 1 is active against mupirocin-resistant staphylococci (n=26) with MICs below 0.5 μg/ml (range ≦0.125-0.5 μg/ml). Moraxella catarrhalis isolates (n=2) are inhibited at MICs of 0.2 and 0.4 μg/ml. The MICs for Enterococcus faecalis isolates (n=2) are 6.4 μg/ml and 12.8 μg/ml. The MIC for Enterococcus faecium (n=1) is 6.4 μg/ml.

The compounds of the present invention are therefore useful for the treatment of microbial, e.g. bacterial diseases, e.g. the treatment of diseases associated with bacterial infections. Treatment includes treatment and prevention (prophylaxis).

In another aspect the present invention provides a compound of the present invention for use as a pharmaceutical, e.g. in the treatment of diseases associated with microbial, such as bacterial infections.

In another aspect the present invention provides the use of a compound of the present invention for the manufacture of a medicament, e.g. in the form of a pharmaceutical composition, for the treatment of a microbial disease, such as bacterial diseases, for example of diseases associated with bacterias such as Staphylococcus spp. and Moraxella catarrhalis.

The compound of example 1 is a preferred compound of the present invention. It has, for example been determined that the minimum inhibitory concentration, e.g. MIC90 (μg/ml), of the compound of Example 1, e.g. in the form of its sodium salt, against, for example S. aureus (MRSA) is of about 0.2. It is therefore, indicated that for the treatment of bacterial diseases, the compounds of the present invention may be administered to larger mammals, for example humans, by similar modes of administration at similar dosages than conventionally used with Linezolid.

In a further aspect the present invention provides a method of treatment of microbial, e.g. bacterial, diseases, e.g. diseases mediated by bacterias such as Staphylococcus spp. and Moraxella, which treatment comprises administering to a subject in need of such treatment an effective amount of a compound of the present invention; e.g. in the form of a pharmaceutical composition, e.g. in combination with another pharmaceutically active agent.

For pharmaceutical use a compound of the present invention includes one or more, preferably one, compounds of the present invention, e.g. a combination of two or more compounds of the present invention.

For such treatment, the appropriate dosage will, of course, vary depending upon, for example, the chemical nature and the pharmakokinetic data of a compound of the present invention employed, the individual host, the mode of administration and the nature and severity of the conditions being treated. However, in general, for satisfactory results in larger mammals, for example humans, an indicated daily dosage is in the range from about 0.01 g to about 1.0 g (from about 1 mg/kg to about 15 mg/kg) of a compound of the present invention; conveniently administered, for example, in divided doses up to four times a day.

A compound of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration;

e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

The compounds of the present invention may be administered in the form of a pharmaceutically acceptable salt, e.g. an acid addition salt or metal salt; or in free form; optionally in the form of a solvate. The compounds of the present invention in the form of a salt exhibit the same order of activity as the compounds of the present invention in free form; optionally in the form of a solvate.

A compound of the present invention may be used for pharmaceutical treatment according to the present invention alone, or in combination with one or more other pharmaceutically active agents. Such other pharmaceutically active agents include other antibactials, e.g. penicillins, cephalosporins, macrolides, vancomycin, rifampicin. Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising a compound of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers,

e.g and further comprising another pharmaceutically active agent.

Such compositions may be manufactured according, e.g. analogously, to a method as conventional, e.g. by mixing, granulating, coating, dissolving or lyophilizing processes. Unit dosage forms may contain, for example, from about 0.5 mg to about 1000 mg, such as 1 mg to about 500 mg.

In the following Examples all temperatures are in degrees Celsius (° C.) and are uncorrected. The following abbreviations are used:
Bn benzyl
BOM benzyloxymethyl
Cbz benzyloxycarbonyl
DMAA N,N-dimethylacetamide
DMAP 4-dimetylaminopyridine
LiHMDS Lithium bis(trimethylsilyl)amide
EDCl N′-(3-dimethylaminopropyl)-N-ethylcarbodiimide in the form of a hydrochloride
EtOAc ethyl acetate
EX Example
PE petrolether
PPTS pyridinium p-toluenesulfonat
rt room temperature
THF tetrahydrofurane
DPM diphenylmethyl

Acremonic acid (also known as Cephalosporin P1) is a compound of formula II_{P}.

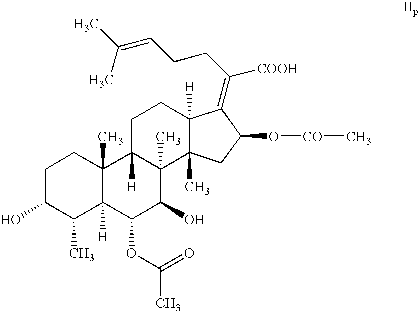

### EXAMPLE 1

### 6-O-(2′-fluoroisobutyryl)-24, 25-dihydro-acremonic acid (compound of formula I, wherein R is —COR₁, wherein R₁ is 2-fluoroisopropyl)

### A. 3-O-Benzyloxymethyl-acremonic acid P1-benzyloxymethylester (compound of formula IV_{P})

9.68 ml of BOM-Cl are added to a solution of 10 g of acremonic acid and 12.2 ml of Hünig's base in 40 ml of anhydrous CH₂Cl₂ at −10°. The reaction mixture obtained is stirred for 15 minutes and allowed to warm up to rt, stirring is continued under argon for 24 hours. H₂O is added to the mixture obtained, the two phases obtained are separated. The organic layer obtained is washed with H₂O, brine and saturated aqueous NaHCO₃-solution, dried and solvent is evaporated. 3-O-Benzyloxymethyl-acremonic acid benzyloxymethylester is obtained.

¹H-NMR (200 MHz, DMSO+D₂O): δ (ppm)=7.32-7.37 (m, 10H, arom.-H), 5.66 (d, J=8.5Hz, H-16), 5.32 (dd, J=6.2Hz, J=16.3Hz, 2H, BOM-CH₂), 5.08 (t, J=6.4Hz, 1H, 24-H), 4.57-4.83 (m, 7H, 6-H, 3×BOM-CH₂), 3.54 (s, 1H, 3-H), 3.34 (s, 1 H, 7-H), 2.00/1.84 (2s, 2×3H, H-34, H-36).

### B. 3-O-Benzyloxymethyl-6-deacetyl-acremonic acid benzyloxymethylester (compound of formula V_{P})

6.97 ml of 2N NaOH are added at 0° to a solution of 11.38 g of 3-O-benzyloxymethyl-acremonic acid benzyloxymethylester in 75 ml of a mixture of THF/MeOH/H₂O=5/4/1. To the reaction mixture obtained 20 ml of THF are added and the solution obtained is stirred at rt for 16 hours. 1.4 ml of 2N NaOH are added to the mixture obtained and solvent is evaporated. The residue obtained is distributed between H₂O and Et₂O, the mixture obtained is extracted, the organic layer obtained is washed with H₂O and brine, dried and solvent is evaporated. 3-O-Benzyloxymethyl-6-deacetyl-acremonic acid benzyloxymethylester is obtained.

¹H-NMR (200 MHz, DMSO): δ (ppm)=7.26-7.32 (m, 10H, arom.-H), 5.68 (d, J=8.2Hz, H-16), 5.32 (dd, J=6.2Hz, J=18.8Hz, 2H, BOM-CH₂), 5.08 (t, 1H, 24-H), 4.47-4.81 (m, 6H, 3×BOM-CH₂), 3.51 (s, 1H, 3-H), 3.49/3.34 (2s, 1H, 6-H, 7-H), 1.85 (1s, 3H, H-34).

¹³C-NMR (50 MHz, DMSO): δ (ppm)=169.56, 168.57, 149.13, 138.18, 137.10, 131.67, 129.09, 128.21, 128.15, 127.66, 127.50, 127.35, 127.27, 123.0, 92.97, 88.18, 82.84, 78.32, 75.84, 73.75, 71.12, 68.62, 49.05, 47.88, 43.55, 42.48, 36.23, 35.66, 30.02, 28.13, 27.89, 25.96, 25.59, 25.38, 22.86, 22.37, 20.75, 20.37, 18.71, 18.32, 17.43.

### C. 3-O-Benzyloxymethyl-6-O-(2′-fluoro-butyryl)-acremonic acid, benzyloxymethylester (compound of formula VI_{P}, wherein R₁ is 2-fluoroisopropyl)

5.02 g of 2-fluoroisobutyric acid are added to a solution of 22.84 g 3-O-benzyloxymethyl-6-deacetyl-acremocic acid, benzyloxymethylester and 3.97 g of DMAP in anhydrous CH₂Cl₂ under argon at 0°. 9.06 g of EDCl are added and the mixture obtained is stirred at rt overnight. The mixture obtained is concentrated and the concentration residue obtained is distributed between EtOAc and H₂O and extracted. The organic layer obtained is washed with H₂O, brine and saturated, aqueous Na₂CO₃-solution, dried and solvent is evaporated. 3-O-Benzyloxymethyl-6-O-(2′-fluoroisobutyryl)-acremonic acid, benzyloxymethylester is obtained.

¹H-NMR (500 MHz, CDCl₃): δ (ppm)=7.35-7.28 (m, 10H, arom.-H), 5.84 (d, 1H, J=8.6Hz, H-16), 5.41/5.27 (2d, J1=J2=6.1Hz, 2H, BOM-CH₂), 5.10 (dt, J=7.2Hz, J=1.3Hz, 1H, 24-H), 4.85-4.83 (m, 1H, BOM-CH₂), 4.73-4.59 (m, 7H, 6-H, 3×BOM-CH₂), 3.62 (d, J=1.8Hz, 1H, 3-H), 3.44 (d, J=2.6Hz, 1H, 7-H), 1.93 (1s, 3H, 34-H), 1.61 (d, J=3.9Hz, 3a′-CH₃), 1.57 (d, J=3.7Hz, 3b′-CH₃).

¹³C-NMR (125 MHz, CDCl₃): δ (ppm)=172.82 (d, J=25Hz, 1′-C), 170.59, 169.22, 148.65, 138.11, 137.02, 132.52, 130.70, 128.44, 128.41, 127.92, 127.80, 127.73, 127.63, 123.12, 93.51, 92.50 (d, J=181Hz, 2′-C), 88.45, 83.53, 80.32, 78.01, 74.27, 71.96, 69.63, 49.71, 48.49, 43.13, 40.92, 39.94, 39.50, 36.62, 35.77, 31.40, 28.83, 28.32, 26.39, 25.99, 25.70, 24.80 (d, J=24Hz, 3a′-C), 24.68 (d, J=24Hz, 3b′-C), 23.71, 23.66, 21.63, 20.78, 18.16, 17.75, 17.21.

### D. 6-O-(2′-fluoroisobutyryl)-24, 25-dihydro-acremonic acid (compound of formula I, wherein R is —COR₁, wherein R₁ is 2-fluoroisopropyl)

20.99 g of 3-O-benzyloxymethyl-6-O-(2′-fluoroisobutyryl)-acremonic acid, benzyloxymethylester are hydrogenated at 1 atm in the presence of Pd(OH)₂/C in 235 ml of a mixture of EtOAc/MeOH=10/1 overnight, the mixture obtained is filtered and solvent is evaporated.

6-O-(2′-fluoroisobutyryl)-24, 25-dihydro-acremonic acid is obtained.

The solid can be recrystallized from cyclohexane/EtOAc: mp=157-160° C.

### EXAMPLE 2

### 3-O-Benzyloxymethyl-6-O-pivaloyl-acremonic acid benzyloxymethylester (compound of formula VI_{P}, wherein R₁ is t.butyl)

1.31 ml of pivaloyl chloride are added at rt to a solution of 5.504 g of 3-O-benzyloxymethyl-6-deacetyl-acremonic acid benzyloxymethylester and 1.13 g of DMAP in anhydrous pyridine under argon. The mixture obtained is stirred under argon at 500 for 20 hours, poured over ice and extracted with EtOAc. The organic layer obtained is washed with H₂O and brine, dried, and solvent is evaporated.

3-O-Benzyloxymethyl-6-O-pivaloyl-acremonic acid benzyloxymethylester is obtained. Splitting off the benzyloxymethyl protecting group and hydrogenation of the double bond is carried out analogously to Example 1, step D.

Analogously to the methods as described in examples 1 and 2, but using appropriate starting materials, compounds of formula I, wherein R is as defined in TABLE 1 below, are obtained. ¹H-NMR data (in DMSO, if not otherwise indicated) of the compounds are also set out in TABLE 1.

TABLE 1

| EX | R | ¹H-NMR |
|---|---|---|
| 1 | 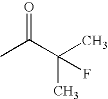 | 5.82 (d, 1H, J=8.7 Hz, 16-H), 4.63 (d, 1H, J=10.6 Hz, 6-H, 3.71(s, 1H, 3-H), 3.43 (s, 1H, 7-H), 1.94 (1s, 3H, 34-H), 1.60 (d, J=4.7 Hz, 3a′-CH₃), 1.55 (d, J=4.6 Hz, 3b′-CH₃) |
| 2 | 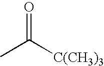 | 5.61 (d, J=8.1 Hz, 1H, 16-H), 4.60 (d, J=9.7 Hz, 1H, 6-H),3.47 (s, 1H, 3-H), 3.19 (s, 1H, 7-H), 1.88 (s, 3H, 34-CH₃),1.13 (s, 9H, 3 × 3′-CH₃) |
| 3 | 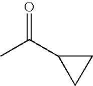 | 5.62 (d, J=8.2 Hz, 1H, 16-H), 4.63 (dd, J=9.7 Hz, J=2.0 Hz,1H, 6-H), 3.47 (s, 1H, 3-H), 3.19 (d, J=2.0 Hz, 1H, 7-H),1.89 (s, 3H, 34-CH₃), 1.21-2.44 (m, 2′-H, 3′-CH₂, 4′-CH₂) |
| 4 | 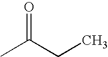 | 5.64 (d, J=7.9 Hz, 1H, 16-H), 4.65 (d, J=10.2 Hz, J=2.0 Hz, 6-H), 3.48 (s, 1H, 3-H), 3.10 (s, 1H, 7-H), 1.92-2.56 (m,9H, cont. 2′-CH₂), 1.89 (s, 3H, 34-CH₃), 1.03 (d, J=7.0 Hz,3 H, 3′-CH₃) |
| 5 | 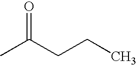 | 5.59 (d, J=8.3 Hz, 1H, 16-H), 4.60 (d, J=9.9 Hz, 1H, 6-H),3.47 (s, 1H, 3-H), 3.30 (s, 1H, 7-H), 1.92-2.56 (m, 9H,cont. 2′-CH₂), 1.89 (s, 3H, 34-CH₃), 0.77-1.80 (m, cont.3′-CH₂, 4′-CH₃) |
| 6 | 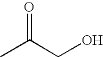 | 5.57 (d, J=8.3 Hz, 1H, 16-H), 4.65 (dd, J=7.9 Hz, J=2.5 Hz,1H, 6-H), 3.93 (dd, J=17.1, J=28.5, 2H, 2′-CH₂), 3.45 (s,1H, 3-H), 3.32 (d, J=2.5 Hz, 1H, 7-H) |
| 7 | 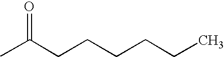 | 5.60 (d, J=8.2 Hz, 1H, 16-H), 4.60 (d, J=10.0 Hz, 1H, 6-H), 3.48 (s, 1H, 3-H), 3.31 (s, 1H, 7-H), 1.92-2.56 (m,9H, cont. 2′-CH₂), 1.89 (s, 3H, 34-CH₃), 0.76-1.80 (m,cont. 3′-CH₂, 4′-CH₂, 5′-CH₂, 6′-CH₂, 7′-CH₃) |
| 8 | 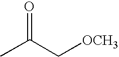 | 5.62 (d, J=6.8 Hz, 1H, 16-H), 4.70 (d, J=9.9 Hz, 1H, 6-H),3.84-4.20 (m, 2H, 2′-CH₂), 3.32-3.44 (m, 5H, 3-H, 7-H,—OCH₃) |
| 9 | 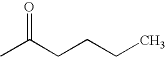 | 5.62 (d, 1H, 16-H), 4.62 (d, 1H, 6-H), 3.45 (s, 1H, 3-H),3.33 (s, 1H, 7-H), 1.92-2.56 (m, 9H, cont. 2′-CH₂), 1.88(s, 3H, 34-CH₃), 0.76-1.80 (m, cont. 3′-CH₂, 4′-CH₂, 5′-CH₃) |
| 10 | 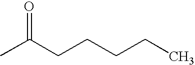 | 5.61 (d, 1H, J=8.2 Hz, 16-H), 4.61 (d, J=9.3 Hz, 1H, 6-H),3.48 (s, 1H, 3-H), 3.31 (s, 1H, 7-H), 1.92-2.56 (m, 9H,cont. 2′-CH₂), 1.87 (s, 3H, 34-CH₃), 0.76-1.80 (m, cont.3′-CH₂, 4′-CH₂, 5′-CH₂, 6′-CH₃) |
| 11 | 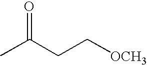 | 5.61 (d, J=8.3 Hz, 16-H), 4.63 (d, J=10.1 Hz, 1H, 6-H), 3.53-3.59 (m, 2H, 2′-CH₂), 3.47 (s, 1H, 3-H), 3.32 (s,1H, 7-H), 3.22 (s, 3H, —OCH₃) |
| 12 | 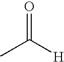 | 8.21 (s, 1H, 1′-H), 5.62 (d, J=7.9 Hz, 1H, 16-H), 4.66 (dd,1H, 6-H), 3.48 (s, 1H, 3-H), 3.38 (d, J=2.8 Hz, 1H, 7-H),1.89 (s, 3H, 34-CH₃) |
| 13 | 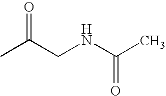 | 8.28 (dd, J=5.8 Hz, 1H, NH), 5.60 (d, J=8.1 Hz, 1H, 16-H),4.64 (dd, J=8.3 Hz, J=2.4 Hz, 1H, 6-H), 3.60 (dd, J=5.8 Hz,2′-CH₂), 3.47 (s, 1H, 3-H), 3.34 (d, J=2.4 Hz, 1H, 7-H),1.85/1.87 (2s, 2 × 3H, 34-CH₃, 4′-CH₃) |
| 14 | 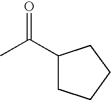 | 5.61 (d, J=7.7 Hz, 1H, 16-H), 4.60 (d, J=9.8 Hz, 1H, 6-H),3.47 (s, 1H, 3-H), 3.25 (s, 1H, 7-H), 2.48-2.84 (m, 2′-H),1.88 (s, 3H, 34-CH₃), 1.00-2.45 (m, 3′-CH₂, 4′-CH₂, 5′-CH₂, 6′-CH₂) |
| 15 | 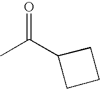 | 5.61 (d, J=8.3 Hz, 1H, 16-H), 4.61 (d, J=10.0 Hz, 1H, 6-H),3.46 (s, 1H, 3-H), 3.27 (s, 1H, 7-H), 3.09 (quin, J=8.3 Hz,2′-H) |
| 16 | 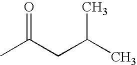 | 5.64 (d, J=8.33 Hz, 1H, 16-H), 4.63 (d, J=10.0 Hz, 1H, 6-H), 3.48 (s, 1H, 3-H), 3.35 (s, 1H, 7-H), 1.95-2.50 (m,cont. 2′-CH₂, 3′-H), 1.90 (s, 3H, 34-CH₃),0.96/0.95/0.93/0.92 (4s, 4 × 4′-CH₃). |
| 17 | 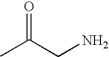 | 5.61 (d, J=8.3 Hz, 1H, 16-H), 4.67 (d, 1H, 6-H), 3.47 (s,1H, 3-H), 3.09-3.40 (m, 5H, 7-H, 2′-CH₂, NH₂) |
| 18 | 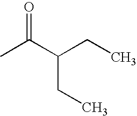 | 5.60 (d, J=8.5 Hz, 1H, 16-H), 4.60 (d, J=10.0 Hz, 1H, 6-H), 3.49 (s, 1H, 3-H), 3.32 (s, 1H, 7-H), 1.87 (s, 3H, 34-CH₃), 1.12/1.00 (2s, 2 × 3H, 4′-CH₃) |
| 19 | 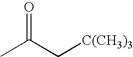 | 5.61 (d, J=7.9 Hz, 1H, 16-H), 4.60 (d, J=10.3 Hz, 1H, 6-H), 3.49 (s, 1H, 3-H), 3.35 (s, 1H, 7-H), 1.89 (s, 3H, 34-CH₃), 1.00 (s, 9H, 4′-CH₃) |
| 20 | 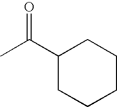 | 5.63 (d, J=8.3 Hz, 1H, 16-H), 4.59 (d, J=9.9 Hz, 1H, 6-H),3.47 (s, 1H, 3-H), 3.24 (s, 1H, 7-H), 0.99-2.35 (m, cont.2′-H, 3′-CH₂, 4′-CH₂, 5′-CH₂, 6′-CH₂, 7′-CH₂) |
| 21 | 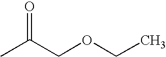 | 5.64 (d, J=8.3 Hz, 1H, 16-H), 4.72 (d, J=8.8 Hz, 1H, 6-H),3.90-4.18 (m, 2H, 2′-CH₂), 3.36-3.57 (m, 4H, 3-H, 7-H,4′-CH₂) |
| 22 | 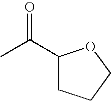 | 5.61 (m, 1H, 16-H), 4.6-4.7 (m, 1H, 6-H), 4.19-4.35 (m,1H, H-2′), 3.80 (m, 2H, 4′-CH₂), 3.34-3.45 (m, 2H, 3-H, 7-H) |
| 23 | 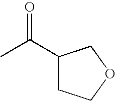 | 5.62 (d, J=7.5 Hz, 1H, 16-H), 4.65 (d, J=9.5 Hz, 6-H),3.03-3.93 (m, 3′-CH₂, 5′-CH₂, 3-H, 7-H) |
| 24 | 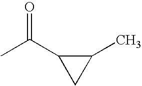 | 5.61 (d, J=8.3 Hz, 1H, 16-H), 4.60 (d, J=11.2 Hz, 6-H),3.47 (s, 1H, 3-H), 3.28 (s, 1H, 7-H), 1.04 (s, 3H, —CH₃(3′-C)) |
| 25 | 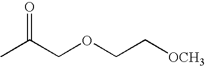 | 5.64 (d, J=8.3 Hz, 1H, 16-H), 4.72 (d, J=8.9 Hz, 1H, 6-H),4.09 (dd, 2H, 2′-CH₂), 3.60-3.64/3.46-3.50 (2m, 5H, 4′-CH₂, 5′-CH₂, 3-H), 3.37 (s, 1H, 7-H), 3.27 (s, 3H, —OCH₃) |
| 26 | 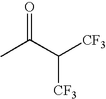 | 5.84 (d, J=8.7 Hz, 1H, 16-H), 4.77 (d, J=10.5 Hz, 6-H),4.10 (sept., 1H, 2′-H), 3.74 (s, 1H, 3-H), 3.50 (s, 1H, 7-H) |
| 27 | 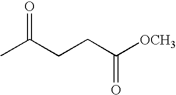 | 5.58 (d, J=8.3 Hz, 1H, 16-H), 4.57 (d, J=10.1 Hz, 6-H),3.57 (s, 3H, —OCH₃), 3.45 (s, 1H, 3-H), 3.29 (s, 1H, 7-H). |
| 28 | 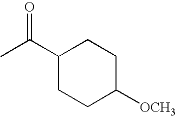 | 5.61 (d, J=8.1 Hz, 1H, 16-H), 4.60 (d, J=9.8 Hz, 1H, 6-H),3.10-3.48 (m, 6H, 5′-H, OCH₃, 3-H, 7-H) |
| 29 | 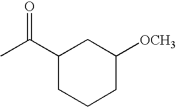 | 5.61 (d, J=8.0 Hz, 1H, 16-H), 4.61 (d, J=10.0 Hz, 6-H), 3.49 (s, 1H, 3-H), 3.05-3.30 (m, 5H, 4′-H, OCH₃, 7-H) |
| 30 | 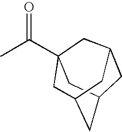 | 5.62 (d, J=8.2 Hz, 1H, 16-H), 4.59 (d, J=9.7 Hz, 1H, 6-H),3.45 (s, 1H, 3-H), 3.17 (d, 1H, 7-H), 1.40-2.45 (m, cont.adamantyl —CH and CH₂) |
| 31 | 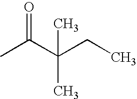 | 5.63 (d, 1H, 16-H), 4.60 (d, J=10.1 Hz, 1H, 6-H), 3.46 (s,1H, 3-H), 3.26 (s, 1H, 7-H), 1.87 (s, 3H, 34-CH₃), 1.00-1.24 (m, cont. 12H, 2 × CH₃(C-2′), 4′-CH₃) |
| 32 | 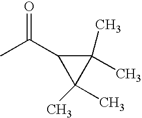 | 5.63 (d, J=8.2 Hz, 1H, 16-H), 4.56 (d, J=10.2 Hz, 6-H),3.45 (s, 1H, 3-H), 3.29 (s, 1H, 7-H), 1.15 (s, 12H, 4 ×—CH₃(3′-C)) |
| 33 | 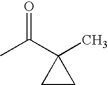 | 5.63 (d, J=8.2 Hz, 1H, 16-H), 4.60 (d, J=10.0 Hz, 6-H),3.45 (s, 1H, 3-H), 3.24 (s, 1H, 7-H), 0.70-1.80 (m, 39H,cont. —CH₃(2′-C), 3′-CH₂, 4′-CH₂) |
| 34 | 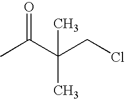 | 5.62 (d, J=8.2 Hz, 1H, 16-H), 4.63 (d, J=9.8 Hz, 1H, 6-H),3.73 (dd, J=10.7 Hz, J=15.9 Hz, 2H, —CH₂Cl), 3.47 (s, 1H,3-H), 3.35 (d, 1H, 7-H), 1.87 (s, 3H, 34-CH₃), 1.20 (s, 6H,2 × CH₃(C-2′) |
| 35 | 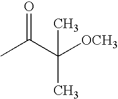 | 5.63 (d, J=8.4 Hz, 1H, 16-H), 4.66 (d, J=10.0 Hz, 1H, 6-H), 3.46 (s, 1H, 3-H), 3.26 (d, 1H, 7-H), 3.16 (s, 3H,OCH₃), 1.87 (s, 3H, 34-CH₃), 1.31 (s, 6H, 2 × CH₃(C-2′) |
| 36 | 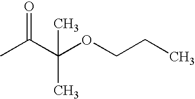 | 5.62 (d, J=8.1 Hz, 1H, 16-H), 4.65 (d, J=9.9 Hz, 1H, 6-H),3.17-3.55 (m, 4H, 3-H, 7-H, —OCH₂), 1.87 (s, 3H, 34-CH₃), 1.32 (s, 6H, 2 × CH₃(C-2′) |
| 37 | 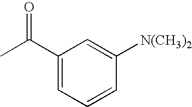 | 7.28-7.38 (m, 3H, arom.H), 6.93 (d, 1H, arom.H), 5.76(d, J=8.6 Hz, 1H, 16-H), 4.79 (d, J=11.2 Hz, 1H, 6-H),3.70 (s, 1H, 3-H), 3.61 (d, 1H, 7-H), 2.98 (s, 6H,—N(CH₃)₂) |
| 38 | 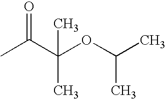 | 5.77 (d, J=8.4 Hz, 1H, 16-H), 4.51 (d, J=10.5 Hz, 1H, 6-H), 3.60-3.74 (m, 2H, 3-H, —OCH(CH₃)₂), 3.34 (s, 1H, 7-H), 1.88 (s, 3H, 34-CH₃), 1.00-1.50 (m, cont., 2 × CH₃(C-2′), —OCH(CH₃)₂) |
| 39 | 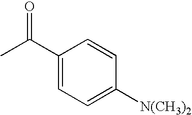 | 7.82 (d, J=8.9 Hz, 2H, arom.H), 6.57 (d, J=8.9 Hz, 2H,arom.H), 5.71 (d, J=8.4 Hz, 1H, 16-H), 4.67 (d, J=10.5 Hz,1H, 6-H), 3.63 (s, 1H, 3-H), 3.52 (d, 1H, 7-H), 2.96 (s,6H, —N(CH₃)₂) |
| 40 | 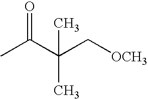 | 5.81 (d, H=8.3 Hz, 1H, 16-H), 4.62 (d, J=10.4 Hz, 1H, 6-H), 3.69 (s, 1H, 3-H), 3.25-3.45 (m, 5H, 7-H, —OCH₃),1.92 (s, 3H, 34-CH₃), 1.16 (s, 6H, 2 × CH₃(C-2′) |
| 41 | 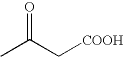 | 5.86 (d, 1H, 16-H), 4.74 (d, J=9.1 Hz, 1H, 6-H), 3.32-3.79(m, 4H, 3-H, 7-H, 2′-CH₂) |
| 42 | 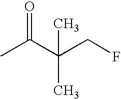 | 5.82 (d, J=8.4 Hz, 1H, 16-H), 4.58 (d, J=10.8 Hz, 1H, 6-H), 4.50/4.26 (ddd, J=8.8 Hz, H=13.2 Hz, J=47.1 Hz, 2H,—CH₂F), 3.70 (s, 1H, 3-H), 3.38 (s, 1H, 7-H), 1.93 (s, 3H,34-CH₃), 1.15-1.21 (m, cont.6H, 2 × CH₃(C-2′) |
| 43 | 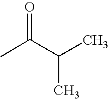 | 5.82 (d, J=8.7 Hz, 1H, 16-H), 4.53 (d, J=10.6 Hz, 1H, 6-H), 3.71 (d, J=2.1 Hz, 1H, 3-H), 3.40 (s, 1H, 7-H), 2.49-2.56 (m, 3H, 13-H, 2′-H, 22a-H), 2.09-2.35 (m, 5H, 22b-H, 5-H, 8-H, 12a-H, 15a-H), 1.94 (s, 3H, 34-CH₃), 1.82-1.89 (m, 3H, 4-H, 2a-H, 11a-H), 1.67-1.72 (m, 3H, 1a-H,12b-H, 2b-H), 1.35-1.53 (m, 6H, 25-H, 11b-H, 23-CH₂,15b-H, 1b-H), 1.14-1.19 (m, 14H, 19-CH₃, 2 × 3′-CH₃, 30-CH₃, 24-CH₂), 1.03 (s, 3H, 18-CH₃), 0.89 (d, J=6.9 Hz,3H, 28-CH₃), 8.86 (d, J=6.9 Hz, 6H, 26-CH₃, 27-CH₃) |
| 44 | 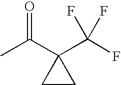 | 5.82 (d, J=8.6 Hz, 1H, 16-H), 4.70 (d, J=10.5 Hz, 1H, 6-H), 3.70 (s, 1H, 3-H), 3.48 (s, 1H, 7-H), 1.94 (s, 3H, 34-CH₃), 1.57-1.28 (m, cont. 4H, 2 × CH₂(cyclopropyl)). |
| 45 | 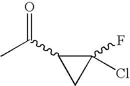 | 5.83 (d, J=8.6 Hz, 1H, 16-H), 4.65-4.58 (m, 1H, 6-H),3.71 (s, 1H, 3-H), 3.56/3.49 (2s, 1H, 7-H), 2.62-2.27 (m,cont. CH(cyclopropyl)), 2.26-2.07/1.95-1.62 (2 m, cont.CH₂(cyclopropyl)). |
| 46 | 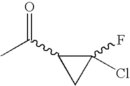 | 5.80 (d, J=8.7 Hz, 1H, 16-H), 4.63 (d, J=10.7 Hz, 1H, 6-H), 3.73 (s, 1H, 3-H), 3.51 (s, 1H, 7-H), 2.61-2.38 (m,cont. CH(cyclopropyl)), 2.38-2.22/2.00-1.63 (2 m, cont.CH₂(cyclopropyl)). |
| 47 | 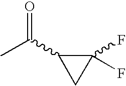 | 5.83 (d, J=8.7 Hz, 1H, 16-H), 4.63 (d, J=10.9 Hz, 1H, 6-H), 3.71 (s, 1H, 3-H), 3.48 (s, 1H, 7-H), 2.51-2.38 (m,cont. 2′-H), 2.09-1.97 (m, 1 H, CHH(cyclopropyl)), 1.96-1.63 (m, cont. CHH(cyclopropyl)). |
| 48 | 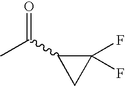 | 5.80 (d, J=8.7 Hz, 1H, 16-H), 4.63 (d, J=10.7 Hz, 1H, 6-H), 3.71 (s, 1H, 3-H), 3.50 (s, 1H, 7-H), 2.50-2.39 (m,cont. 2′-H), 2.20-2.00 (m, cont. CHH(cyclopropyl)), 1.86-1.75 (m, cont. CHH(cyclopropyl)). |
| 49 | 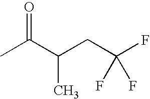 | 5.85-5.82 (m, 1H, 16-H), 4.57-4.53 (m, 1H, 6-H), 3.71 (s,1H, 3-H), 3.46/3.41 (2 s, 2 × 1H, 7-H), 2.82 (m, cont.—OCOCH(CH₃)CHHCF₃), 2.49-2.05 (m, cont.—OCOCH(CH₃)CHHCF₃), 1.32-1.23 (m, cont.—OCOCH(CH₃)CHHCF₃). |

The compounds of examples 2, 30, 31 and 34 in TABLE 1 are obtained analogously as described on Example 2, but using appropriate starting materials; all other compounds of TABLE 1 are obtained analogously as described in Example 1, but using appropriate starting materials. The compounds of examples 1, 2, 34, 42 and 43 are also obtained in the form of a sodium salt.

### EXAMPLE 50

### 6-O-Methyl-24, 25-dihydro-acremonic acid (compound of formula I_{P}, wherein R is methyl)

### A. 3-O-Benzyloxymethyl-6-O-methyl-24, 25-dihydro-acremonic acid diphenylmethylester

0.67 ml of LiHMDS (1M in THF) are added to a solution of 500 mg of 3-O-Benzyloxymethyl-6-deacetyl-acremonic acid diphenylmethylester (which may be obtained according to a method as described in reaction A in example 1 but using appropriate starting materials) in 5 ml of dry N,N-dimethylformamide at −10° and to the mixture obtained 0.06 ml of CH₃I are added after 10 minutes. The mixture obtained is stirred at rt for 2 hours and poured onto ice. The mixture obtained is extracted 3 times with EtOAc. The organic layer obtained is dried, solvent is evaporated and the evaporation residue obtained is subjected to chromatography. 3-O-Benzyloxymethyl-6-O-methyl-acremonic acid diphenylmethylester is obtained.

### B. 6-O-Methyl-24, 25-dihydro-acremonic acid

241 mg of 3-O-benzyloxymethyl-6-O-methyl-acremonic acid diphenylmethylester are hydrogenated at 1 atm in the presence of Pd(OH)₂/C in 3 ml of EtOAc overnight, the mixture obtained is filtered, solvent is evaporated and the evaporation residue is subjected to chromatography. 6-O-methyl-24, 25-dihydro-acremonic acid is obtained.

Analogously as described in Example 50, but using appropriate starting materials, compounds of formula I, wherein R is as defined in TABLE 2 below, are obtained. ¹H-NMR data (in DMSO, if not otherwise indicated) of the compounds are also set out in TABLE 2.

TABLE 2

| EX | R | ¹H-NMR |
|---|---|---|
| 50 | 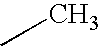 | 5.65 (d, J=8.3 Hz, 1H, 16-H), 3.34-3.53 (m, 2H, 3-H, 7-H),3.19 (s, 3H, —OCH₃), 2.80 (d, 1H, J=9.6 Hz, 6-H) |
| 51 | 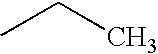 | 5.66 (d, 1H, 16-H), 3.21-3.65 (m, 4H, —OCH₂, 3-H, 7-H),2.91 (d, 1H, 6-H) |
| 52 | 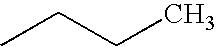 | 5.64 (d, J=8.3 Hz, 1H, 16-H), 3.14-3.53 (m, 4H, —OCH₂, 3-H, 7-H), 2.88 (d, J=9.5 Hz, 1H, 6-H) |
| 53 | 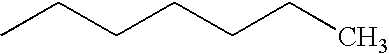 | 5.54 (d, J=8.4 Hz, 1H, 16-H), 3.08-3.40 (m, 4H, —OCH₂, 3-H, 7-H), 2.76 (d, J=9.2 Hz, 1H, 6-H) |

In TABLE 3 below there are listed mass spectroscopy data of intermediates of formula

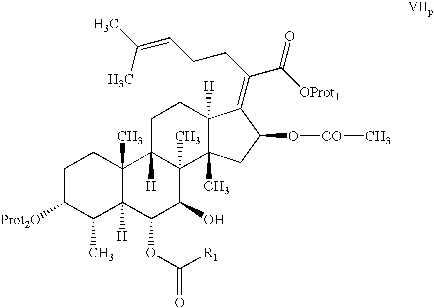

wherein R₁ is as defined in TABLE 3, useful in the production of a compound of formula I_{P}. The numbers in column “EX”, marked with an apostroph (e.g. 1′), are intermediates used in the production of a the corresponding compound of formula I_{P} in TABLE 1. E.g. the intermediate “1′” in TABLE 3 is the intermediate used in the production of the compound of Example 1 in TABLE 1. Mass spectroscopy data (m/z (ESI)), also set out in TABLE 3, are determined by a Finnigan Navigator ThermoQuest LC/MS system.

TABLE 3

| EX | Prot₂ | R₁ | Prot₁ | m/z (ESI) |
|---|---|---|---|---|
| 1′ | BOM | 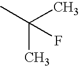 | BOM | [M + Na]⁺ = 883.4 |
| 2′ | BOM | 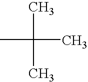 | BOM | [M + Na]⁺ = 879.5 |
| 3′ | BOM | 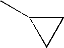 | DPM | [M + Na]⁺ = 909.4 |
| 4′ | H | 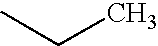 | DPM | [M + Na]⁺ = 777.3 |
| 5′ | H | 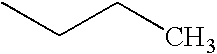 | DPM | [M + Cl]⁻ = 803.4 |
| 6′ | BOM | 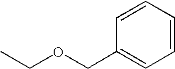 | DPM | [M]⁺ = 989.4 |
| 7′ | BOM | 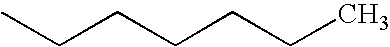 | DPM | [M + Na]⁺ = 953.5 |
| 8′ | BOM | 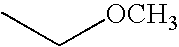 | DPM | [M + Na]⁺ = 914.1 |
| 9′ | BOM | 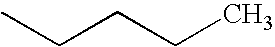 | DPM | [M + Na]⁺ = 925.0 |
| 10′ | BOM | 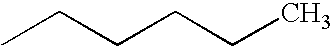 | DPM | [M + Na]⁺ = 939.2 |
| 11′ | BOM | 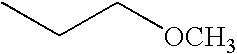 | DPM | [M + Na]⁺ = 926.9 |
| 12′ | BOM | hydrogen | DPM | [M + Na]⁺ = 869.0 |
| 13′ | BOM | 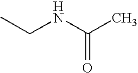 | DPM | [M + Cl]⁻ = 951.9 |
| 14′ | BOM | 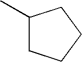 | DPM | [M + Na]⁺ = 937.0 |
| 15′ | BOM | 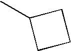 | DPM | [M + Na]⁺ = 923.0 |
| 16′ | BOM | 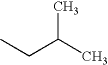 | DPM | [M + Na]⁺ = 925.0 |
| 17′ | BOM | 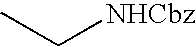 | DPM | [M + Na]⁺ = 1031.8 |
| 18′ | BOM | 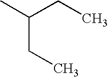 | DPM | [M + Na]⁺ = 938.8 |
| 19′ | BOM | 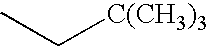 | DPM | [M + Na]⁺ = 939.0 |
| 20′ | BOM | 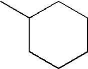 | DPM | [M + Na]⁺ = 951.0 |
| 21′ | BOM | 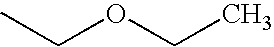 | DPM | [M + Na]⁺ = 927.1 |
| 22′ | BOM | 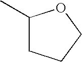 | DPM | [M + Na]⁺ = 939.3 |
| 23′ | BOM | 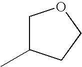 | DPM | [M + Na]⁺ = 939.3 |
| 24′ | BOM | 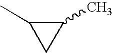 | DPM | [M + Na]⁺ = 923.2 |
| 25′ | BOM | 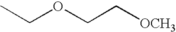 | BOM | [M + Na]⁺ = 911.5 |
| 26′ | BOM | 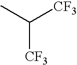 | BOM | [M]⁺ = 950.4 |
| 27′ | BOM | 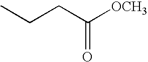 | BOM | [M + Na]⁺ = 909.2 |
| 28′ | BOM | 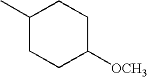 | BOM | [M + Na]⁺ = 935.5 |
| 29′ | BOM | 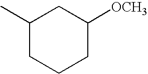 | BOM | [M + Na]⁺ = 935.5 |
| 30′ | BOM | 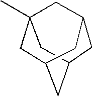 | BOM | [M + Na]⁺ = 957.6 |
| 31′ | BOM | 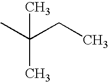 | BOM | [M + Na]⁺ = 894.0 |
| 32′ | BOM | 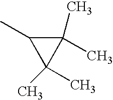 | BOM | [M + Na]⁺ = 919.0 |
| 33′ | BOM | 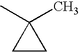 | BOM | [M + Na]⁺ = 877.4 |
| 34′ | BOM | 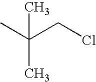 | BOM | [M + Na]⁺ = 913.4 |
| 35′ | BOM | 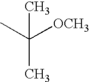 | BOM | [M + Na]⁺ = 895.4 |
| 36′ | BOM | 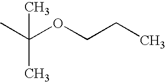 | BOM | [M + Na]⁺ = 921.6 |
| 37′ | BOM | 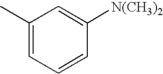 | BOM | [M + Na]⁺ = 942.5 |
| 38′ | BOM | 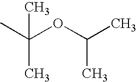 | BOM | [M + Na]⁺ = 923.7 |
| 39′ | BOM | 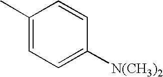 | BOM | [M + Na]⁺ = 942.5 |
| 40′ | BOM | 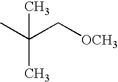 | BOM | [M + Na]⁺ = 909.5 |
| 41′ | BOM | 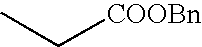 | BOM | [M + Na]⁺ = 972.2 |
| 42′ | BOM | 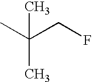 | BOM | [M + Na]⁺ = 897.8 |
| 43′ | BOM | 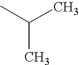 | BOM | [M + Na]⁺ = 865.5 |
| 44′ | BOM | 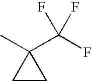 | BOM | [M + Na]⁺ = 931.5 |
| 45′ | BOM | 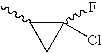 | BOM | [M + Na]⁺ = 915 |
| 46′ | BOM | 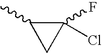 | BOM | [M + Na]⁺ = 915 |
| 47′ | BOM | 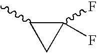 | BOM | [M + Na]⁺ = 899.5 |
| 48′ | BOM | 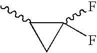 | BOM | [M + Na]⁺ = 899.5 |
| 49′ | BOM | 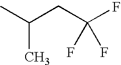 | BOM | [M + Na]⁺ = 933.4 |

In TABLE 4 below there are listed mass spectroscopy data of intermediates of formula

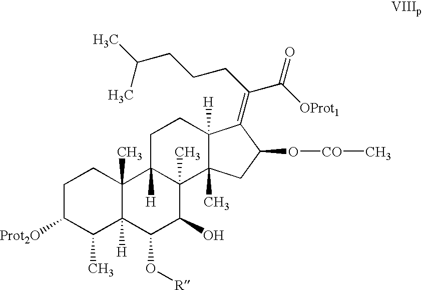

wherein R″ is as defined in TABLE 4, useful in the production of a compound of formula I_{P}. The numbers in column “EX”, marked with an apostroph (e.g. 50′), are intermediates used in the production of a the corresponding compound of formula I_{P} in TABLE 2. E.g. the intermediate “50′” in TABLE 4 is the intermediate used in the production of the compound of Example 50 in TABLE 2. Mass spectroscopy data (m/z (ESI)), also set out in TABLE 4, are determined by a Finnigan Navigator ThermoQuest LC/MS system.

TABLE 4

| EX | Prot₂ | R″ | Prot₁ | m/z (ESI) |
|---|---|---|---|---|
| 50′ | BOM | methyl | DPM | [M + Na]⁺ = 855.1 |
| 51′ | BOM | ethyl | DPM | [M + Na]⁺ = 869.4 |
| 52′ | BOM | n-propyl | DPM | [M + Na]⁺ = 883.3 |
| 53′ | BOM | n-hexyl | DPM | [M + Na]⁺ = 925.5 |

1. 2-(16-Acetoxy-3,7-dihydroxy-4,8,10,14-tetramethyl-6-[hydroxy, (C₁₋₂₂)alkoxy or carbonyloxy]-hexadecahydro-cyclopenta[a]phenanthren-17-ylidene)-6-methyl-heptanoic acids.

2. A The compound of the formula 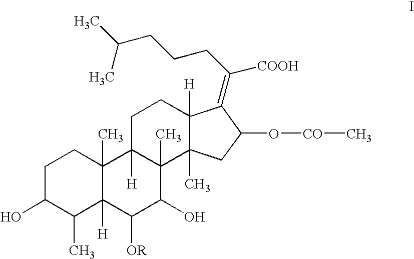
wherein
R is hydrogen, CO—R₁ or (C₁₋₂₂)alkyl, and
R₁ is hydrogen, (C₁₋₂₂)alkyl, (C₃₋₈)cycloalkyl, (C₁₋₆)alkoxy-(C₁₋₆)alkyl, (C₁₋₄)alkoxy-(C₁₋₄)alkoxy-(C₁₋₄)alkyl, amino(C₁₋₄)alkyl, halo(C₁₋₆)alkyl, hydroxy(C₁₋₄)alkyl, hydrogencarbonyl, hydroxycarbonyl(C₁₋₄)alkyl, (C₁₋₄)alkoxy-carbonyl-(C₁₋₄)alkyl, (C₆₋₁₈)aryl, heterocyclyl having 5 or 6 ring members and 1 to 4 heteroatoms selected from S, O or N, or bridged (C₇₋₁₂)cycloalkyl.

3. A compound of claim 2, wherein
R is hydrogen, (C₁₋₆)alkyl, or CO—R₁, and
R₁ is hydrogen, (C₁₋₆)alkyl, (C₃₋₆)cycloalkyl, (C₁₋₃)alkoxy-(C₁₋₃)alkyl, methoxy-(C₁₋₂)alkoxy-(C₁₋₂)alkyl, aminomethyl, halo(C₁₋₄)alkyl comprising one or two halogen atoms, hydroxymethyl, hydroxycarbonylmethyl, methoxycarbonyl-(C₁₋₂)alkyl, phenyl, tetrahydrofuranyl or adamantanyl.

4. A compound of claim 2, wherein R is a group of formula

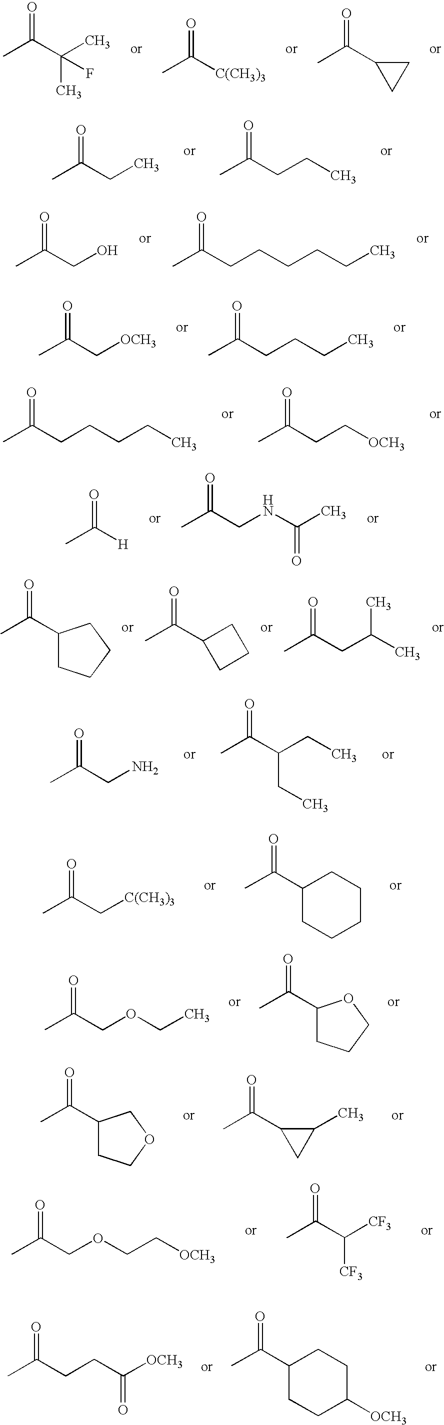

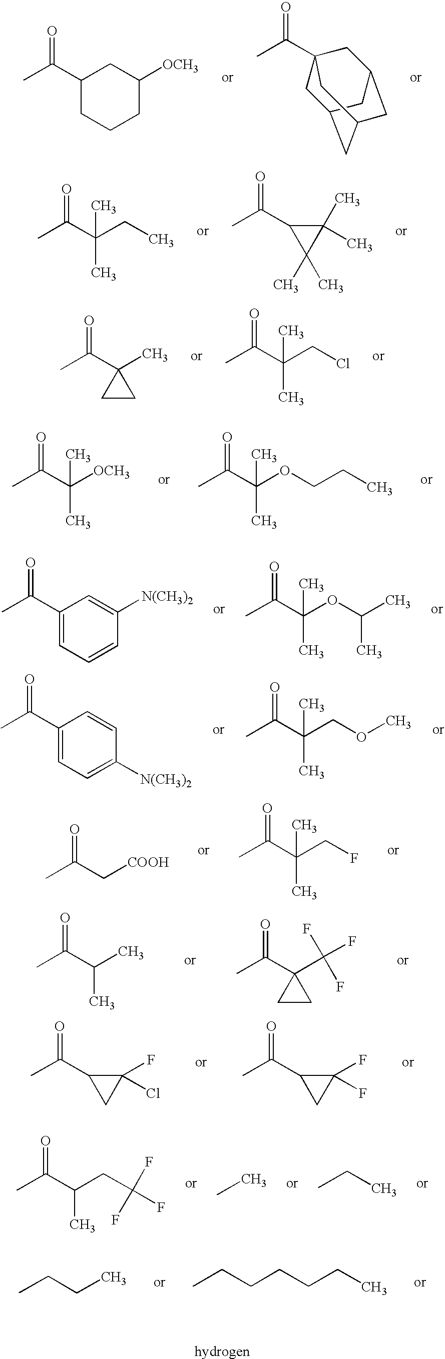

5. The compound of claim 2 in the form of a salt.

6. (canceled)

7. (canceled)

8. A pharmaceutical composition comprising a compound of claim 2 in association with at least one pharmaceutical excipient.

9. The pharmaceutical composition according to claim 8, further comprising another pharmaceutically active agent.

10. A method of treating microbial diseases, comprising administering to a subject in need of such treatment an effective amount of a compound of claim 2.

11. A compound of formula 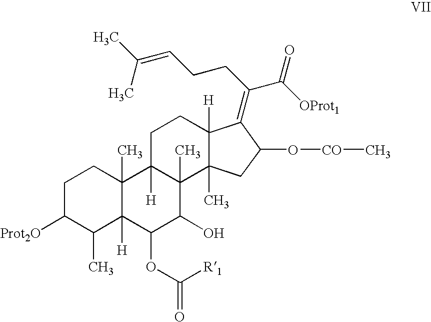
wherein
Prot₁ is a protecting group, and
Prot₂ is either other than a protecting group, or is a protecting group, and
R′₁ has the meaning of R₁, wherein R₁ is hydrogen, (C₁₋₂₂)alkyl, (C₃₋₈)cycloalkyl, (C₁₋₆)alkoxy-(C₁₋₆)alkyl, (C₁₋₄)alkoxy-(C₁₋₄)alkoxy-(C₁₋₄)alkyl, amino(C₁₋₄)alkyl, halo(C₁₋₆)alkyl, hydroxy(C₁₋₄)alkyl, hydrogencarbonyl, hydroxycarbonyl(C₁₋₄)alkyl, (C₁₋₄)alkoxy-carbonyl-(C₁₋₄)alkyl, (C₆₋₁₈)aryl, heterocyclyl having 5 or 6 ring members and 1 to 4 heteroatoms selected from S, O or N, or bridged (C₇₋₁₂)cycloalkyl, and additionally includes residues as defined in R₁, wherein functional groups are protected.

12. A compound of formula 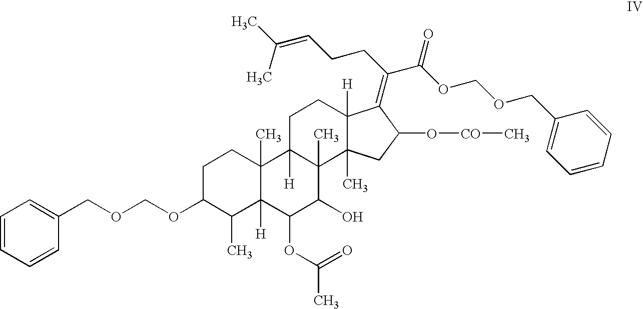
or of formula

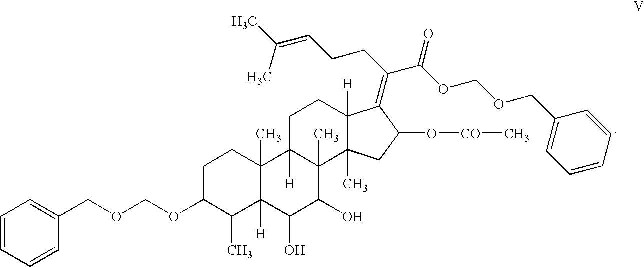

13. A compound of formula 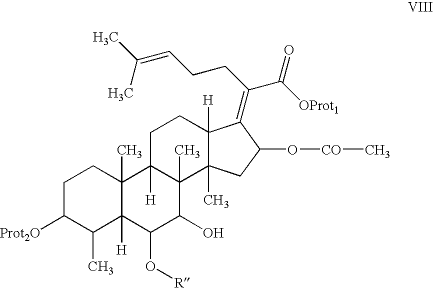
wherein
Prot₁ is a protecting group, and
Prot₂ is either other than a protecting group, or is a protecting group, and R″ is (C₁₋₈)alkyl.
